(19) United States
(12) Patent Application Publication
(10) Pub. No.: US 2005/0261540 A1
(43) Pub. Date: Nov. 24, 2005

(54) Syringe handling device

(76) Inventors: Douglas D. Dischino, Middlefield, CT (US); Richard C. Carlson, Milford, CT (US); James J. Mongillo, Northford, CT (US); Christopher J. Bernard, Cheshire, CT (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 11/121,149

(22) Filed: May 3, 2005

Related U.S. Application Data
(60) Provisional application No. 60/573,672, filed on May 21, 2004.

(51) Int. Cl. A61M 3/00

(52) U.S. Cl. 600/4

(57) ABSTRACT

A syringe handling device for handling a syringe having a syringe needle, a syringe barrel defining a syringe cavity and a syringe plunger at least partially disposed within the syringe cavity is provided and includes a syringe support device, wherein the syringe support device includes at least one of a needle support structure and a barrel support structure and at least one actuation device, wherein the at least one actuation device includes at least one of a decapper actuation device, a plunger actuation device and a lock actuation device.

### CROSS-REFERENCE TO RELATED APPLICATIONS

This application claims the benefit of U.S. Provisional Application Ser. No. 60/573,672 filed May 21, 2004.

### FIELD OF THE INVENTION

This disclosure relates generally to an apparatus for handling a syringe and more particularly to an apparatus for remotely handling a syringe containing a radioactive material in a controlled manner.

### BACKGROUND OF THE INVENTION

As pharmaceutical development and disease diagnostic techniques advance and improve, the advantages that nuclear medicine has over conventional medical techniques for certain applications are becoming more apparent. As such, the use of radioactive substances, such as radionuclides, for detecting tumors, irregular/inadequate blood flow to various tissues and inadequate functioning of organs has increased in popularity. To date, a variety of nuclear imaging techniques exist and include Positron Emission Tomography (PET), Single Photon Emission Computed Tomography (SPECT), Cardiovascular Imaging and Bone Scanning.

For some applications nuclear imaging techniques are superior to conventional imaging techniques. For example, Positron Emission Tomography (PET) is a high resolution, non-invasive, imaging technique which uses the decaying properties of a radionuclide to visualize disease in living tissue. As such, PET imaging is a valuable tool for studying subjects, such as primates, for the development of pharmaceuticals to treat a variety of health conditions. During the PET procedure, a radionuclide is used to produce a plurality of radioactive particles for detection by the PET device. A radionuclide is an unstable substance which emits subatomic particles (e.g. beta particles, alpha particles, neutrons, positrons and/or photons) as it decays, wherein the type of subatomic particles emitted is dependent upon the type of radionuclide. For example, fluorine-18 (F-18), which emits β+ particles and has a half-life (t ½) of 110 minutes, is one of the most widely used positron-emitting nuclides in a clinical setting.

As the F-18 decays, a positively charged electron, called a positron, is emitted from the nucleus with a kinetic energy of several hundred KeV. Each positron then travels a finite distance before interacting with an electron from a different atom to form a transient species called a positronium ion. The positronium ion then undergoes annihilation producing two photons, or gamma rays, each of which have an energy equal to 511 keV and a nearly opposing direction of motion (180° from each other).

PET imaging systems typically have a ring of detectors (scintillators) that encircle the subject that is being imaged. Because each annihilation event creates two 511 keV photons traveling in opposite directions, coincidence detection circuits record only those photons that are detected simultaneously by two detectors located on opposing sides of the subject. The number of such simultaneous events indicates the number of positron annihilation events that occurred along a line joining the two opposing detectors. Typically, within a few minutes, hundreds of millions of events are recorded to indicate the number of annihilation events along lines joining pairs of detectors in the ring. These numbers are then used to create a high resolution image using well known tomography techniques.

One problem that currently exists with working with radioactive materials, such as F-18 or 99^{m}Tc-Cardolite, involves the radiation exposure received by the scientists working with these materials. Unlike patients who may only be exposed to a source of radiation infrequently throughout their lifetime, those individuals who receive daily exposure to radiation, such as a radiochemist and/or a radiopharmacist, are at a far greater risk for developing health problems. This is because these substances emit an ionizing radiation. As such, when this radiation interacts with the atoms of a living subject, orbital electrons surrounding these atoms can be ‘knocked’ off by the collisions with the emitted particles. It is well known that the ‘loss’ of an electron from atoms in living tissue can cause health and development problems for that tissue ranging from cell death to genetic mutation leading to birth defects and/or cancer. Thus, the only known way to work with these substances and avoid health consequences is to eliminate or reduce exposure of the radiochemist to the ionizing radiation. In fact, the actions of those involved in the routine handling of radioactive materials are guided by the ALARA recommendation of the Nuclear Regulatory Commission which states that at all times exposure to radioactive material should be As Low As Reasonably Achievable. One way to reduce exposure is by working with these substances while they are disposed in containers shielded with lead. For example, radiation emitted from F-18 requires a lead shield of approximate two inches in width to contain the emitted radiation.

Unfortunately, however, this method does not totally eliminate the radiation exposure to the radiochemist. This is because the radioactive material must be handled and prepared before it can be used. Historically only large medical centers, universities or national laboratories equipped with a cyclotron to produce the positron-emitting radioisotope and PET cameras were involved in the synthesis and utilization of these short lived radionuclides. In these situations, the ¹⁸F produced in the cyclotron target would be transferred via tubing directly into a hot cell where the radiosynthesis of compounds occurred via a synthesis station. Following high performance liquid chromatography purification and subsequent formulation, this material would then be available for clinical studies. Recently however, there has been the advent of cyclotron-free PET imaging centers. This has been made possible by the creation of regional production facilities which are responsible for the synthesis, purification and distribution of ¹⁸F labeled compounds, primarily ¹⁸F-FDG (¹⁸F-fluorodeoxyglucose). These facilities arrange for land transportation of the radiolabeled product suitable for human use to cyclotron-free PET imaging centers, which can be as far as 100-150 miles from the production facility.

Using the same model, cyclotron-free radiosynthesis facilities have been created in private industry for the purpose of preparing proprietary radiolabeled compounds for drug discovery and development operations. In this situation, a typical scenario may be as follows. The aqueous ¹⁸F is obtained directly from the cyclotron target and is contained within a glass vial or, most commonly in a needle capped syringe (typically 1 mL or 3 mL). The aqueous ¹⁸F is then shipped via a lead shipping container (often referred to as a pig) to a customer for use. Upon receipt of the radioactive material, the syringe containing the radioactive material is removed from the lead shipping pig and lowered into a dose calibrator to measure the amount of radioactivity which will be used in the synthesis. Once the radioactivity has been measured, the syringe is removed from the dose calibrator, the needle cap is removed from the syringe needle and the radioactive aqueous solution is dispensed into a lead shielded reaction vial by manually actuating the syringe plunger. The lead shielded reaction vial is then placed into a synthesis system which is typically located within a hot cell.

Although steps are taken to shield the radiochemist in order to reduce the overall exposure to radiation, certain body parts still experience a higher than desired level of exposure. Specifically, the fingers and hands of the radiochemist still experience a higher than desired level of exposure because the protective cap of the needle must be removed manually and the aqueous solution must be manually dispensed into the glass reaction vial.

Another problem that exists when working with radioactive materials involves the accidental release of the radioactive solution. It is well known that human interaction in the handling of radioactive materials increases the likelihood of accidents and spills. The above steps provide for the opportunity of radioactive spillage from an inadvertent engagement of the needle and the syringe or worse, an accidental contact of the exposed tip of the needle with the gloved hands of the radiochemist with possible puncture of the skin. As such, the removal of the human element from this interaction would greatly aid in reducing the likelihood of accidental exposure.

### SUMMARY OF THE INVENTION

A syringe handling device for handling a syringe having a syringe needle, a syringe barrel defining a syringe cavity and a syringe plunger at least partially disposed within the syringe cavity is provided and includes a syringe support device, wherein the syringe support device includes at least one of a needle support structure and a barrel support structure and at least one actuation device, wherein the at least one actuation device includes at least one of a decapper actuation device, a plunger actuation device and a lock actuation device.

A method for handling a syringe using a syringe handling device is provided, wherein the syringe handling device includes a syringe support structure and at least one of a plunger actuation device and a decapper actuation device. The method includes associating the syringe with the syringe support structure such that the syringe is associated with at least one of the plunger actuation device and the decapper actuation device and operating the syringe handling device such that the syringe and at least one of the plunger actuation device and the decapper actuation device interact with each other.

A machine-readable computer program code is provided wherein the program code includes instructions for causing a controller to implement a method for handling a syringe using a syringe handling device, wherein the syringe handling device includes a syringe support structure and at least one of a plunger actuation device and a decapper actuation device. The method includes associating the syringe with the syringe support structure such that the syringe is associated with at least one of the plunger actuation device and the decapper actuation device and operating the syringe handling device such that the syringe and at least one of the plunger actuation device and the decapper actuation device interact with each other.

### BRIEF DESCRIPTION OF DRAWINGS

The foregoing and other features and advantages of the present invention will be more fully understood from the following detailed description of illustrative embodiments, taken in conjunction with the accompanying drawings in which:

FIG. 1 is a top down view of syringe handling device, in accordance with an exemplary embodiment;

FIG. 2 is a top down view of a needle cradle of the syringe handling device of FIG. 1;

FIG. 3 is a front view of the needle cradle of the syringe handling device of FIG. 1;

FIG. 4 is a top down view of a barrel cradle of the syringe handling device of FIG. 1;

FIG. 5 is a side view of the barrel cradle of the syringe handling device of FIG. 1;

FIG. 6 is a front view of the barrel cradle of the syringe handling device of FIG. 1;

FIG. 7 is a top down view of a lock actuation device of the syringe handling device of FIG. 1 in a disengaged configuration;

FIG. 8 is a top down view of a locking mechanism of the syringe handling device of FIG. 1;

FIG. 9 is a front view of the locking mechanism of the syringe handling device of FIG. 1;

FIG. 10 is a side view of a lock actuation device of the syringe handling device of FIG. 1;

FIG. 11 is a side view of a lock actuation member of the syringe handling device of FIG. 1;

FIG. 12 is a top down view of a lock actuation device of the syringe handling device of FIG. 1 in an engaged configuration;

FIG. 13 is a top down view of a needle cradle, barrel cradle and locking actuation device of the syringe handling device of FIG. 1, with the locking actuation device in an engaged configuration;

FIG. 14 is a top down view of a needle cradle, barrel cradle and locking actuation device of the syringe handling device of FIG. 1, with the locking actuation device in a disengaged configuration;

FIG. 15 is a side view of the plunger actuation device of the syringe handling device of FIG. 1, with the plunger actuation device in a retracted configuration;

FIG. 16 is a side view of a plunger actuation member of a plunger actuation device of the syringe handling device of FIG. 1;

FIG. 17 is a side view of a plunger actuator of a plunger actuation device of the syringe handling device of FIG. 1;

FIG. 18 is a side view of a plunger mechanism of a plunger actuation device of the syringe handling device of FIG. 1;

FIG. 19 is a side view of the plunger actuation device of the syringe handling device of FIG. 1, with the plunger actuation device in an extended configuration;

FIG. 20 is a top down view of the syringe handling device of FIG. 1, with the locking actuation device and the plunger actuation device in a disengaged and retracted configuration, respectively;

FIG. 21 is a top down view of the syringe handling device of FIG. 1, with the locking actuation device and the plunger actuation device in an engaged and extended configuration, respectively;

FIG. 22 is a side view of a decapper actuation device of the syringe handling device of FIG. 1 in an extended configuration;

FIG. 23 is a side view of a decapper actuation device of the syringe handling device of FIG. 1 in a retracted configuration;

FIG. 24 is a top down view of a decapper actuation device of the syringe handling device of FIG. 1 in an extended configuration;

FIG. 25 is a top down view of a decapper actuation device of the syringe handling device of FIG. 1 in a retracted configuration;

FIG. 26 is a top down view of the needle cradle, barrel cradle and decapper actuation device of the syringe handling device of FIG. 1, with the decapper actuation device in an extended configuration;

FIG. 27 is a top down view of the needle cradle, barrel cradle and decapper actuation device of the syringe handling device of FIG. 1, with the decapper actuation device in a retracted configuration;

FIG. 28 is a block diagram illustrating a method for implementing the syringe handling device of FIG. 1;

FIG. 29 is a side view of a syringe with the plunger pulled out of the barrel;

FIG. 30 is a side view of the syringe of FIG. 28 with the plunger pushed into the barrel;

FIG. 31 is a top down view of the syringe handling device of FIG. 1, with a capped syringe disposed within the syringe handling device;

FIG. 32 is a top down view of the syringe handling device of FIG. 1, with a capped syringe disposed within the syringe handling device and with locking mechanism engaged;

FIG. 33 is a top down view of the syringe handling device of FIG. 1, with a capped syringe disposed within the syringe handling device, with the locking mechanism engaged and with the decapper actuator retracted; and

FIG. 34 is a top down view of the syringe handling device of FIG. 1, with a decapped syringe disposed within the syringe handling device, with the locking mechanism engaged, the decapper actuator retracted and the plunger actuator extended.

### DETAILED DESCRIPTION OF THE INVENTION

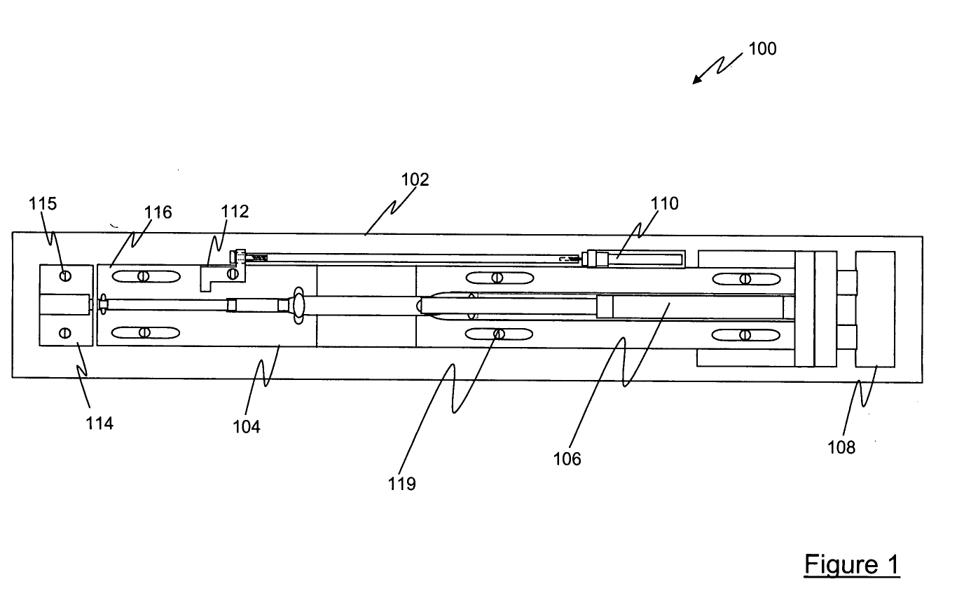

Referring to FIG. 1, a syringe handling device 100 is illustrated and includes a device base 102, a syringe nest 104, a plunger actuation device 106, a decapper actuation device 108 and a lock actuation device 110 having a locking mechanism 112. Syringe nest 104 includes needle support structure or needle cradle 114 and a barrel support structure or barrel cradle 116, wherein barrel cradle 116 is movably associated with device base 102 and needle cradle 114. Although in the disclosed embodiment the syringe nest 104 is shown as including both the needle cradle 114 and the barrel cradle 116, embodiments having only one of the needle cradle 114 and/or the barrel cradle 116 are contemplated. Also, although in the disclosed embodiment the barrel cradle 116 is shown as being movably associated with the device base 102 and the needle cradle 114, it is contemplated that the needle cradle 114 may also be movably associated with the barrel cradle 116.

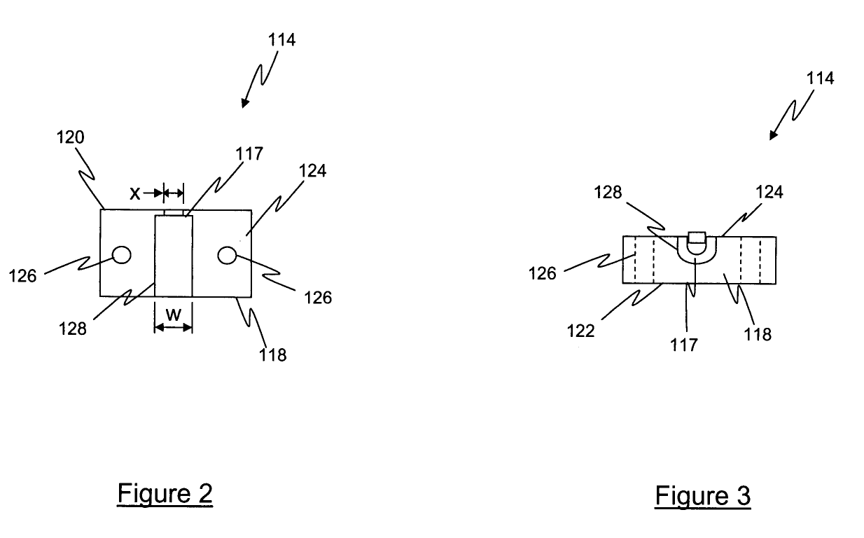

Referring to FIG. 2 and FIG. 3, needle cradle 114 includes a needle cradle front surface 118, a needle cradle rear surface 120, a needle cradle bottom surface 122 and a needle cradle top surface 124. Needle cradle 114 defines at least one needle cradle mounting cavity 126, which traverses the width of needle cradle 114 such that needle cradle top surface 124 is communicated with needle cradle bottom surface 122.

Needle cradle top surface 124 defines a needle cradle channel 128 which communicates needle cradle front surface 118 with needle cradle rear surface 120. Needle cradle channel 128 includes a first needle channel diameter w and a second needle channel diameter x, wherein first needle cradle channel diameter w is larger than second needle cradle channel diameter x to form a channel lip 117.

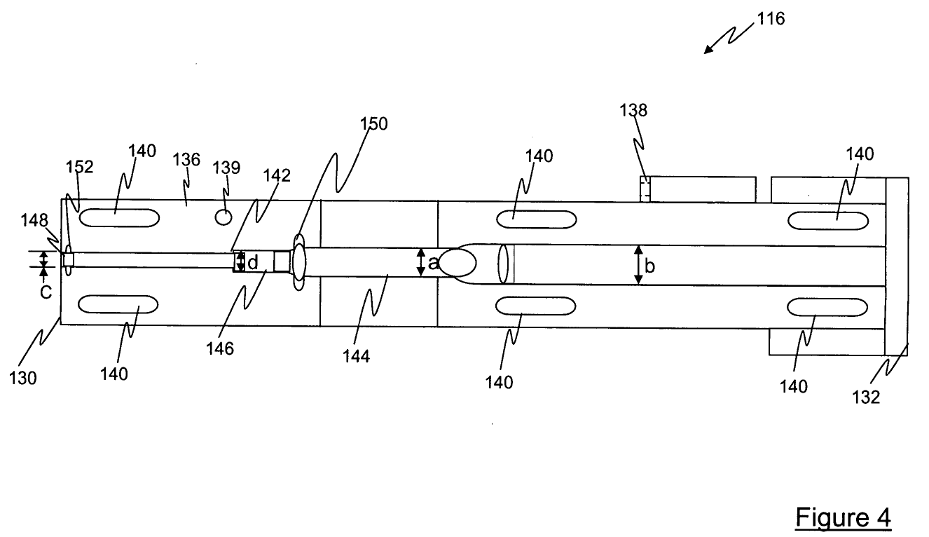

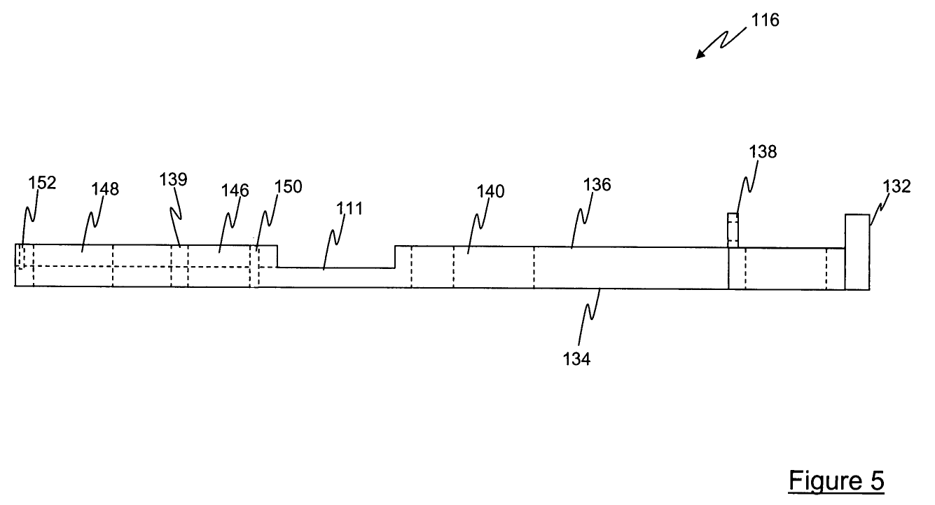

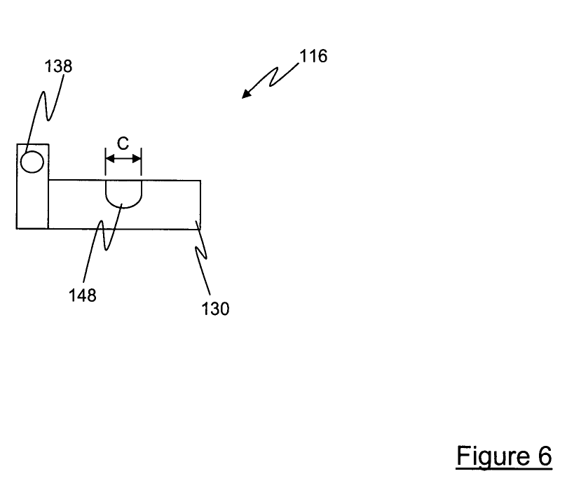

Referring to FIG. 4, FIG. 5 and FIG. 6, barrel cradle 116 includes a barrel cradle front surface 130, a barrel cradle rear surface 132, a barrel cradle bottom surface 134, a barrel cradle top surface 136 and a locking actuation device support structure 138. Barrel cradle 116 defines at least one threaded cavity 139 and at least one elongated guide cavity 140 which traverses the width of barrel cradle 116 such that barrel cradle top surface 136 is communicated with barrel cradle bottom surface 134.

Barrel cradle 116 defines a plunger cavity 144 and a barrel channel 142 having an aft barrel channel 146 and a forward barrel channel 148, wherein aft barrel channel 146 communicates forward barrel channel 148 with plunger cavity 144. Plunger cavity 144 includes a first plunger cavity width a, a second plunger cavity width b and at least one finger press channel 150. Aft barrel channel 146 includes an aft barrel channel diameter d and forward barrel channel 148 includes a forward barrel channel diameter c, wherein forward barrel channel 148 also includes at least one barrel notch 152 disposed in adjacent proximity to the barrel cradle front surface 130. It should be appreciated that aft barrel channel diameter d may be sized similarly to forward barrel channel diameter c or may be sized to accommodate the barrel of a needle and an adhesive label documenting the contents of the barrel. Moreover, the first plunger cavity width a may be sized to accommodate the plunger of a needle and a plunger gripping device which may be used to insert and remove the needle from barrel cradle 116.

Referring back to FIG. 1, needle cradle 114 is non-movably associated with device base 102 via at least one mounting screw 115 and barrel cradle 116 is movably associated with device base 102 via at least one guide screw 119, wherein guide screw 119 may be used to movably associate barrel cradle 116 with device base 102 such that barrel cradle 116 is allowed to traverse laterally along the surface of device base 102 a predetermined distance as allowed by guide cavity 140.

Although guide cavity 140 is shown as being sized and shaped to allow barrel cradle 116 to move using a guide screw 119, it is contemplated that barrel cradle 116 may be movably mounted using any method suitable to the desired end purpose, such as a snap-on guide rail. Moreover, although syringe handling device 100 is illustrated and discussed as having a syringe nest 104 movably mounted to a device base 102, it should be considered within the scope of the disclosed embodiments that the syringe nest 104 may be a standalone unit and/or that the needle cradle 114 may be movably associated with the barrel cradle 116.

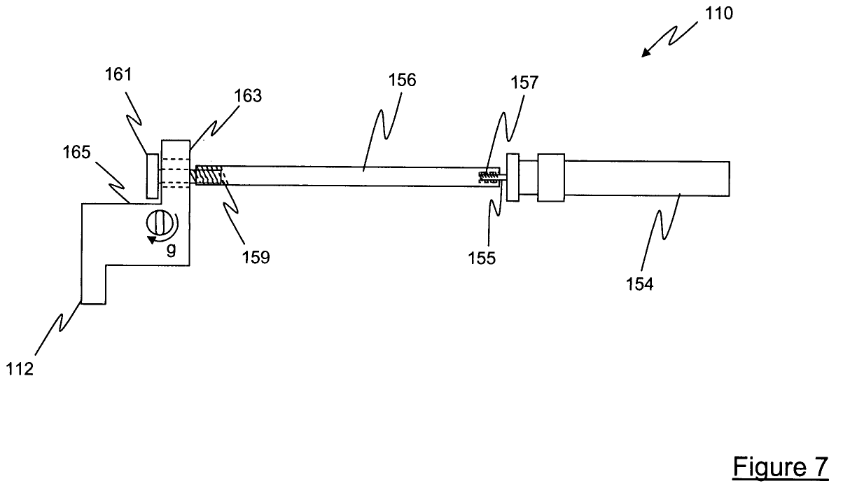

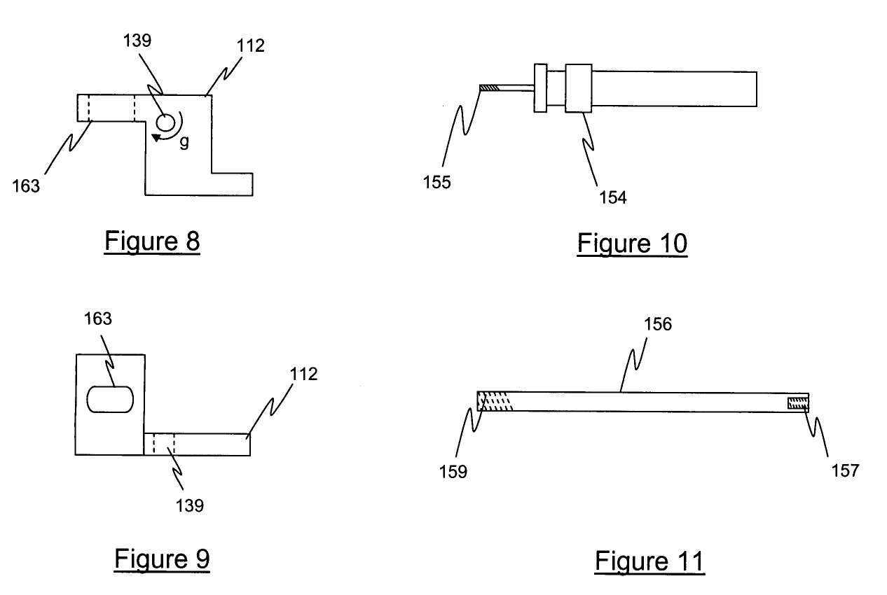

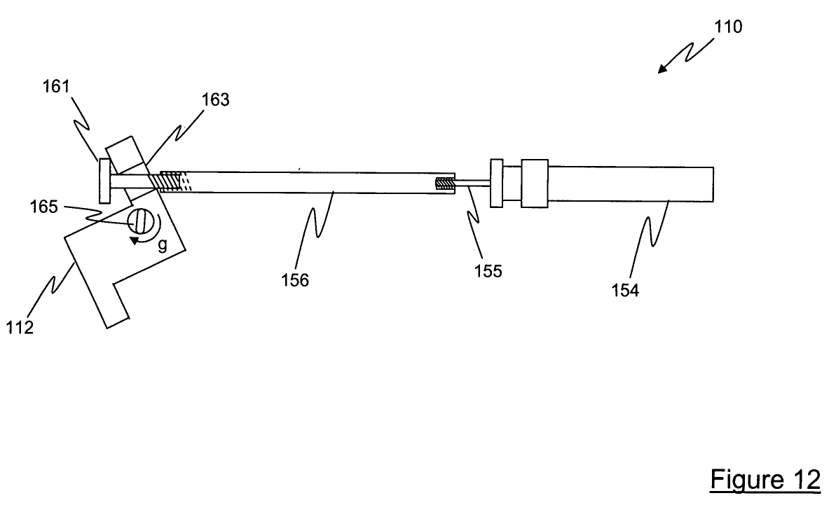

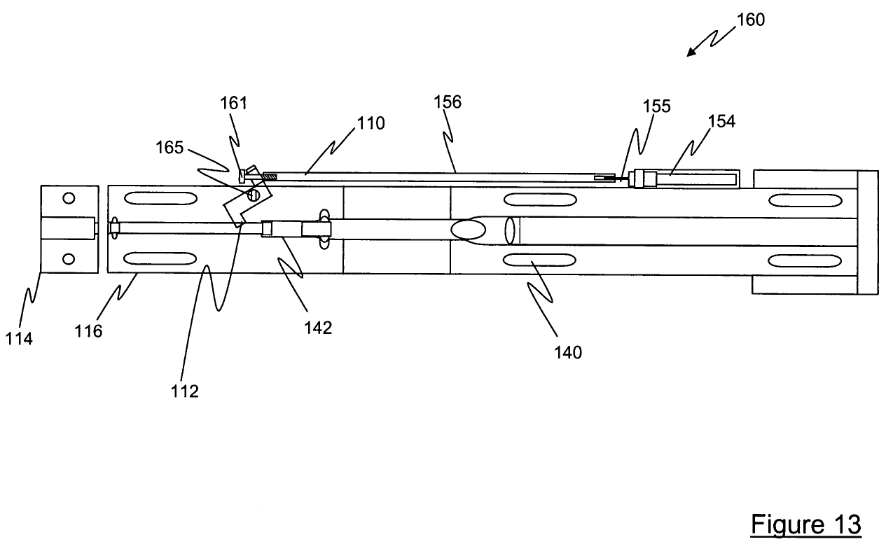

Referring to FIGS. 7-14, lock actuation device 110 is shown and includes a lock actuator 154 and a lock actuation member 156, wherein lock actuator 154 includes a threaded actuator portion 155 and is associated with locking mechanism 112 via lock actuation member 156. Lock actuation member 156 includes a first threaded portion 157 and a second threaded portion 159, wherein first threaded portion 157 interacts with threaded actuator portion 155 to associate lock actuation member 156 with lock actuator 154. Moreover, lock actuation member 156 is movably associated with locking mechanism 112 via a threaded mechanism screw 161 which interacts with second threaded portion 159 via a locking mechanism guide cavity 163.

Although, lock actuator 154 is shown as being non-movably associated with barrel cradle 116 via a press-fit friction fit with locking actuation device support structure 138, lock actuator 154 may be non-movably associated with barrel cradle 116 via any method suitable to the desired end purpose, such as a clip, an adhesive and/or a screw. Moreover, locking mechanism guide cavity 163 may be sized and shaped to allow threaded mechanism screw 161 to move in a lateral (side to side) manner relative to locking mechanism 112 as lock actuation device 110 is configured between a disengaged configuration 158 and an engaged configuration 160. Furthermore, although locking mechanism 112 is shown as a mechanism that covers at least a portion of the barrel channel 142, locking mechanism 112 may be any mechanism which interacts with at least one of the syringe nest 104 and the syringe to securely associate the syringe with at least one of the barrel cradle 116 and the needle cradle 114.

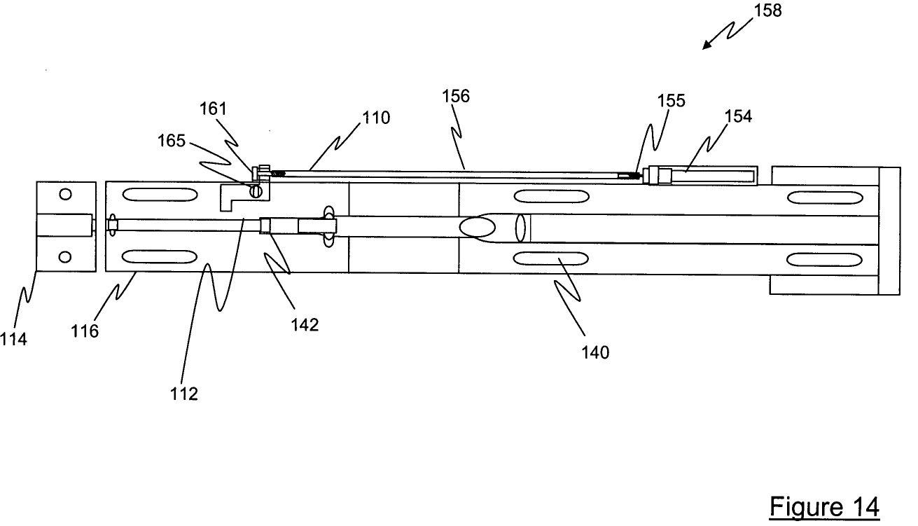

As mentioned above, lock actuation device 110 is configurable between disengaged configuration 158 and engaged configuration 160, wherein locking mechanism 112 is movably associated with barrel cradle 116 via a mounting screw 165 threadingly associated with threaded cavity 139 such that locking mechanism 112 rotates about an axis g traversing axially through threaded cavity 139. As such, when lock actuation device 110 is configured from the engaged configuration 160 into the disengaged configuration 158, lock actuator 154 interacts with lock actuation member 156 to cause locking mechanism 112 to rotate about its axis g such that locking mechanism 112 is disposed away from forward barrel channel 148, as shown in FIG. 7 and FIG. 14. When lock actuation device 110 is configured from the disengaged configuration 158 into the engaged configuration 160, lock actuator 154 interacts with lock actuation member 156 to cause locking mechanism 112 to rotate about its axis g such that at least a portion of locking mechanism 112 is disposed over barrel channel 142 to partially cover barrel channel 142.

When a syringe is disposed within syringe handling device 100 and lock actuation device 110 is in the engaged configuration 160, locking mechanism 112 is disposed over barrel channel 142 to contain the syringe within the syringe handling device 100. Configuring lock actuation device 110 from the engaged configuration 160 into the disengaged configuration 158 causes locking mechanism 112 to be disposed away from barrel channel 142, allowing the syringe to be removed from the syringe handling device 100.

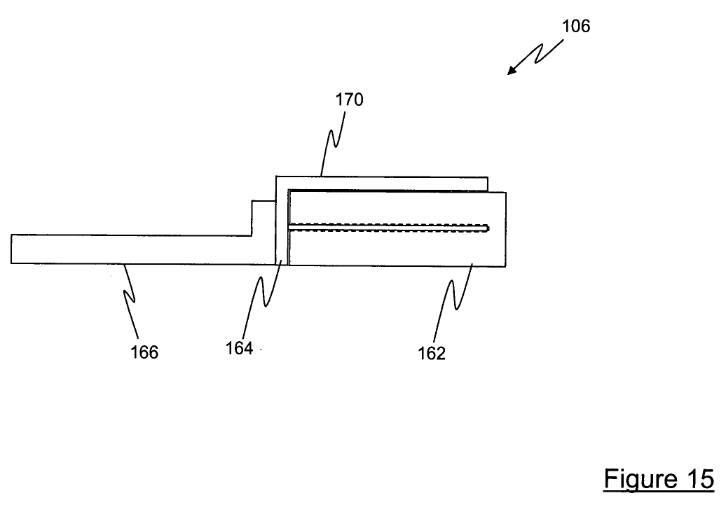

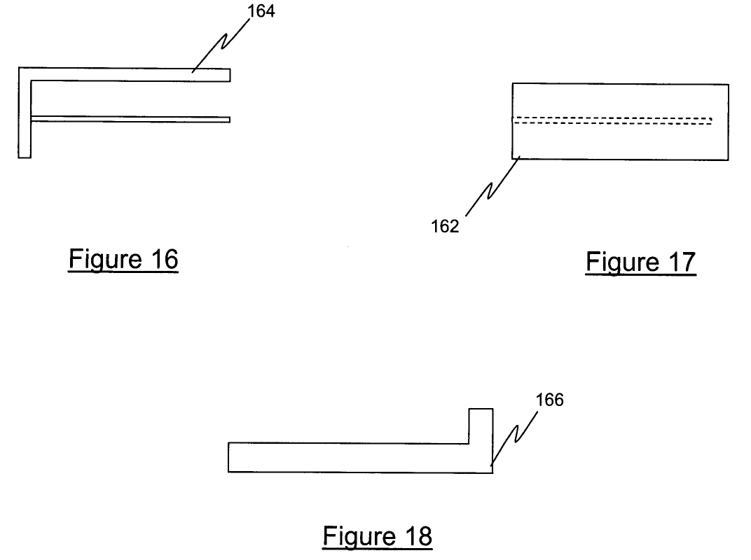

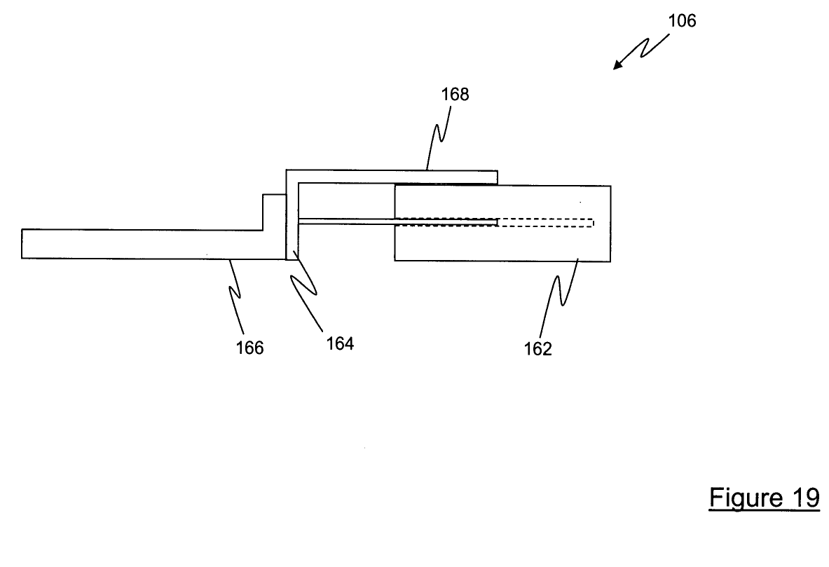

Referring to FIGS. 15-19, plunger actuation device 106 is shown and includes a plunger actuator 162, a plunger actuation member 164 and a plunger mechanism 166, wherein plunger actuator 162 is associated with plunger mechanism 166 via plunger actuation member 164 and wherein plunger actuation device 106 is configurable between an extended configuration 168 and a retracted configuration 170. When plunger actuation device 106 is being configured from the retracted configuration 170 into the extended configuration 168, plunger actuator 162 causes plunger actuation member 164 to extend away from plunger actuator 162 toward plunger mechanism 166. This causes plunger mechanism 166 to move laterally away from plunger actuator 162. When plunger actuation device 106 is being configured from the extended configuration 168 into the retracted configuration 170, plunger actuator 162 causes plunger actuation member 164 to move toward plunger actuator 162, thereby causing the plunger mechanism 166 to move laterally toward the plunger actuator 162.

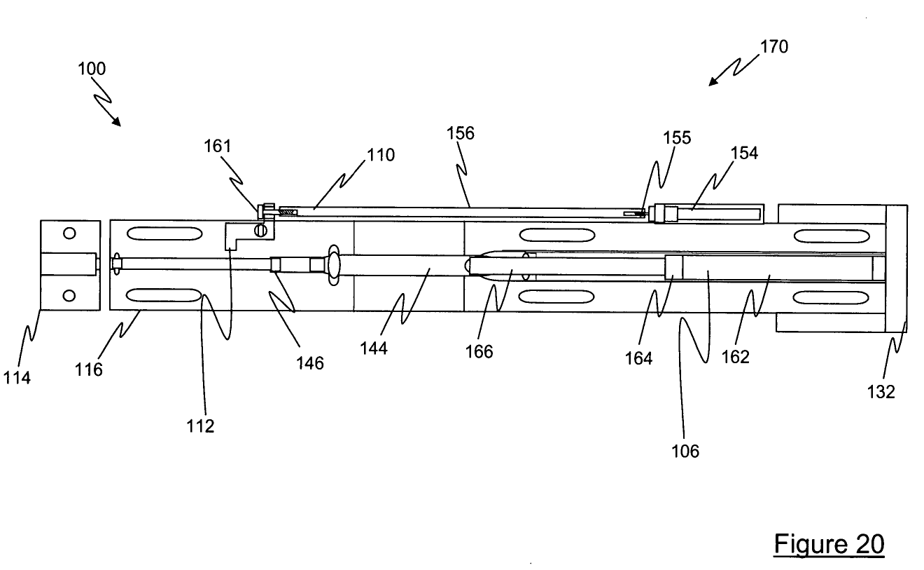

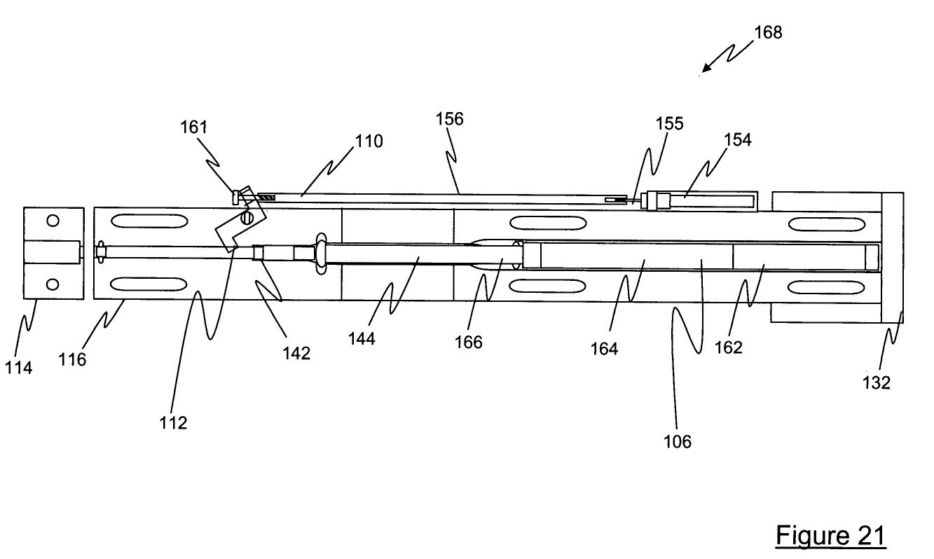

Referring to FIG. 20 and FIG. 21, plunger actuation device 106 is shown disposed within plunger cavity 144, such that plunger mechanism 166 is disposed closest to aft barrel channel 146 and plunger actuator 162 is disposed closest to barrel cradle rear surface 132. Plunger actuator 162 may be associated with syringe nest 104 via any method suitable to the desired end purpose, such as a clip, a screw, a bolt, an adhesive and/or a friction fit. When a syringe is disposed within syringe handling device 100 and plunger actuation device 106 is in retracted configuration 170, the syringe plunger is able to be fully extended such that the barrel of the syringe is able to contain a substance. When plunger actuation device 106 is configured from retracted configuration 170 into extended configuration 168, plunger mechanism 166 is pushed away from plunger actuator 162 such that plunger mechanism 166 traverses plunger cavity 144 in the direction of the needle cradle 114. As the syringe plunger traverses the length of the plunger cavity 144, and hence the syringe barrel, any substance contained within the syringe barrel is dispensed through the syringe needle. It should be appreciated that the plunger mechanism 166 may be connected to the syringe plunger such that that syringe plunger may be controllably contracted into and/or extended away from the syringe barrel via the plunger mechanism 166.

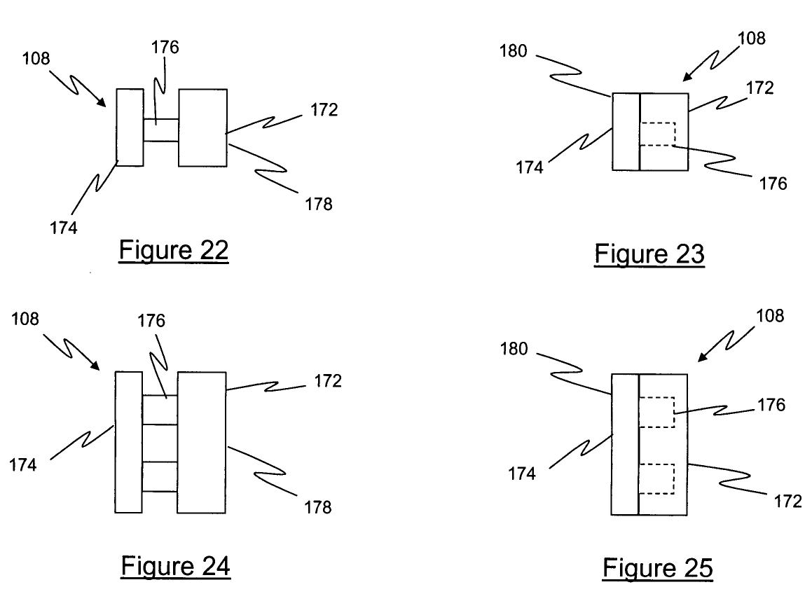

Referring to FIG. 1 and FIGS. 22-25, decapper actuation device 108 is shown and includes a decapper actuator 172 and a decapper mechanism 174 non-movably associated with barrel cradle rear surface 132, wherein decapper actuator 172 is associated with decapper mechanism 174 via a decapper actuation member 176 and wherein decapper actuation device 108 is configurable between an extended configuration 178 and a retracted configuration 180.

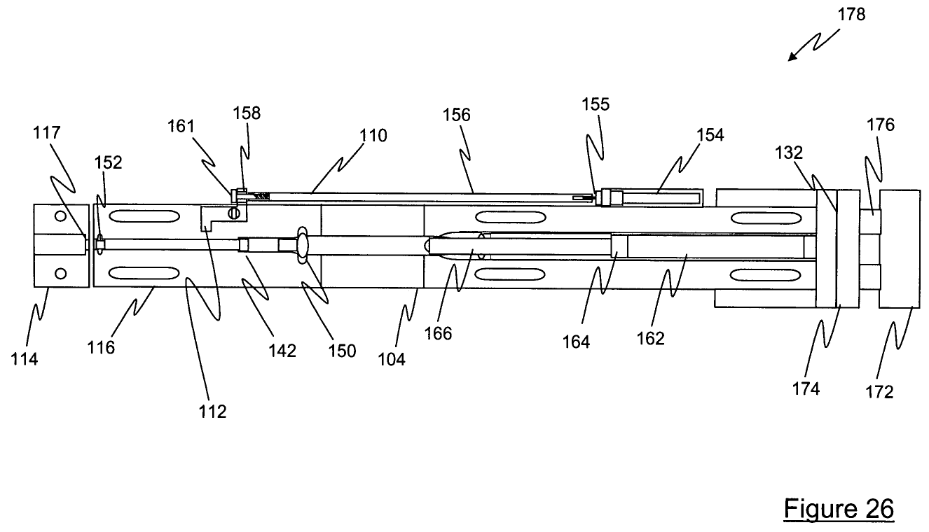

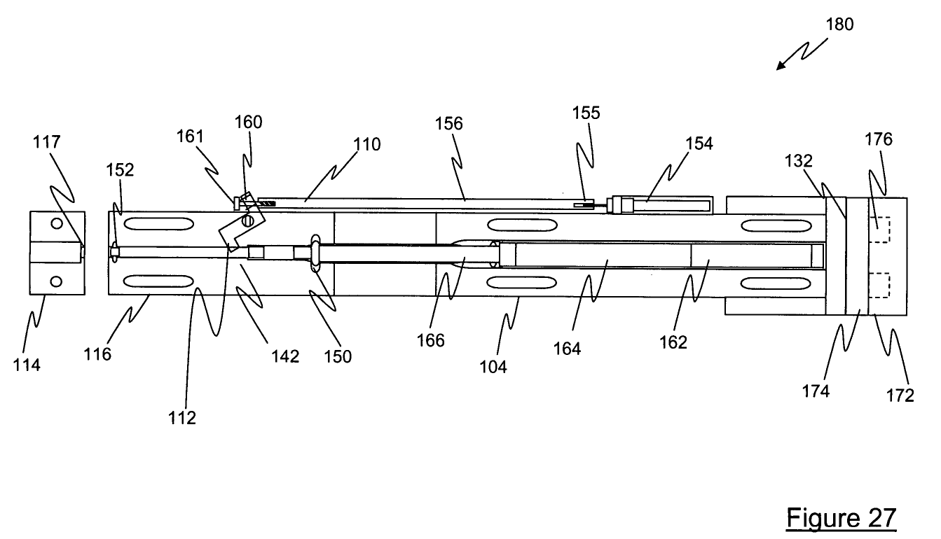

Referring to FIG. 26 and FIG. 27, when a capped syringe is disposed within syringe nest 104, the syringe needle is disposed within needle cradle 114 and barrel cradle 116 such that protruding portions of the syringe needle are disposed within barrel notches 152 of barrel cradle 116 and such that the syringe barrel is disposed within barrel cradle 116 so that the protruding finger press portions of the syringe barrel are disposed within finger press channels 150. When lock actuation device 110 is configured into engaged configuration 160, this operates to anchor the syringe barrel and the syringe needle within the syringe handling device 100. As such, the syringe cap is disposed within needle cradle channel 128 to be associated with first needle channel diameter w, wherein the diameter of the syringe cap is larger than second needle cradle channel diameter x. As the decapper actuation device 108 is being configured from the extended configuration 178 into the retracted configuration 180, decapper actuator 172 causes decapper actuation member 176 and hence, barrel cradle 116, to move toward decapper actuator 172 and away from needle cradle 114. This causes channel lip 117 to push against the syringe cap resulting in the syringe cap being removed from the syringe needle.

The syringe handling device 100 allows for the controlled and remote handling of a syringe containing a radioactive substance. Syringe handling device 100 allows for a syringe to be disposed and contained within syringe nest 104 via locking mechanism 112, the syringe cap to be removed via decapper mechanism 174 and for the substance contained within the syringe barrel to be dispensed via plunger actuation member 164. As such, a method for implementing the syringe handling device 100 is discussed below. However, it should be appreciated that other embodiments are considered to be within the scope of the embodiments disclosed herein. For example, a syringe may be non-movably disposed and contained with the syringe nest 104 via a containment mechanism that frictionally interacts with the syringe to allow the syringe plunger to be extended and/or contracted and/or to allow the syringe cap to be removed.

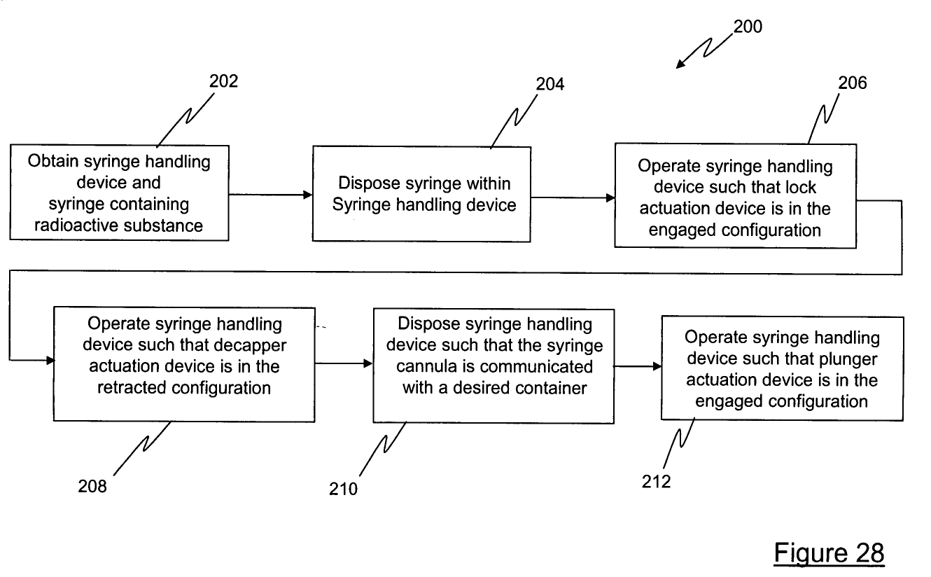

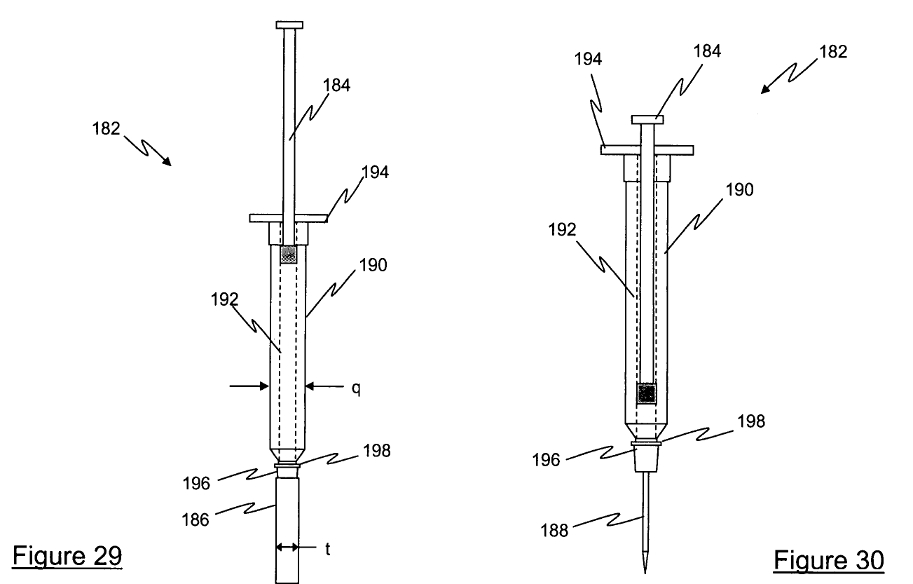

Referring to FIG. 28, a block diagram illustrating a method 200 for implementing syringe handling device 100 is shown and discussed. As shown in block 202, a syringe handling device 100 and a syringe is obtained. FIG. 29 illustrates a syringe 182 having a syringe plunger 184 disposed in its decompressed configuration and FIG. 30 illustrates syringe 182 with syringe plunger 184 in its compressed configuration. As can be seen, syringe 182 includes a needle cap 186 disposed over a syringe needle 188. Syringe 182 also includes a syringe barrel 190 defining a syringe barrel cavity 192 and having a syringe barrel diameter q, wherein syringe plunger 184 is movably associated with syringe barrel cavity 192. Syringe barrel 190 also includes at least one finger press portion 194 which allows a user to apply a force between syringe plunger 184 and syringe barrel 190 in the direction of syringe needle 188. This allows syringe plunger 184 to be configured between a decompressed configuration and a compressed configuration such that the syringe plunger 184 traverses the length of syringe barrel cavity 192. Syringe needle 188 includes a needle base 196 having a needle base lip 198 and is shown as being associated with syringe barrel 190 via a friction fit. Needle cap 186 includes a needle cap diameter t, wherein needle cap diameter t is larger than second needle cradle channel diameter x. Moreover, syringe barrel diameter q is smaller than barrel channel diameter c.

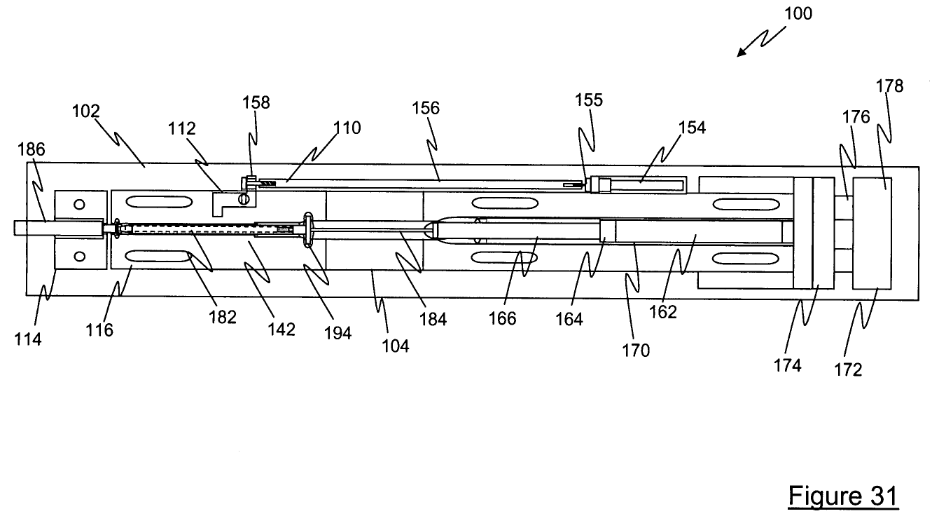

Syringe 182 may be disposed within syringe nest 104, as shown in block 204 and FIG. 31, such that needle cap 186 is disposed within needle cradle channel 128 to be associated with first needle cradle channel diameter w and such that syringe barrel 190 is disposed within forward barrel channel 148 and aft barrel channel 146. Because syringe barrel diameter q is smaller than barrel channel diameter c, syringe 182 is disposed lower than barrel cradle top surface 136. Moreover, syringe 182 is disposed within syringe nest 104 such that finger press portion 194 is disposed within finger press channels 150, needle base lip 198 is disposed within plurality of barrel notches 152 and needle cap 186 is disposed adjacent to channel lip 117. It should be appreciated that syringe 182 may be disposed within syringe nest 104 using any positioning device suitable to the desired end purpose such as a processor controlled positioning device, a manually controlled positioning device and/or a gripping device, such as a commercially purchased flexible multi-pronged plunger gripping device. In this situation, the positioning device may hold the syringe 182 by the syringe plunger 184 or any other suitable syringe structure to place the syringe 182 into the syringe nest 104.

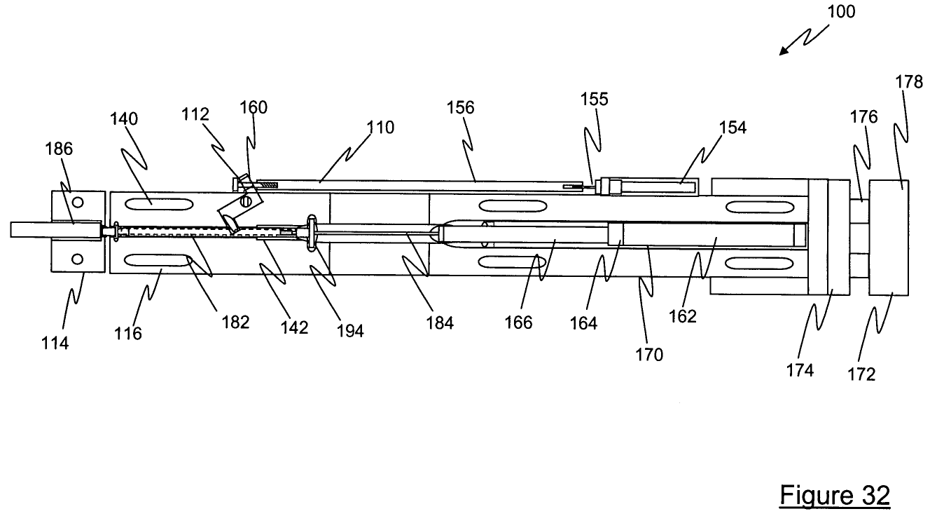

Syringe handling device 100 may be operated, as shown in block 206 and FIG. 32, such that lock actuation device 110 is configured into engaged configuration 160. As explained above, this may be accomplished by causing lock actuator 154 to apply a force to lock actuation member 156 in the direction of locking mechanism 112. This causes locking mechanism 112 to rotate about it axis g such that locking mechanism 112 is disposed over and at least partially covering barrel channel 142 and syringe 182 causing syringe 182 to be retained within syringe nest 104.

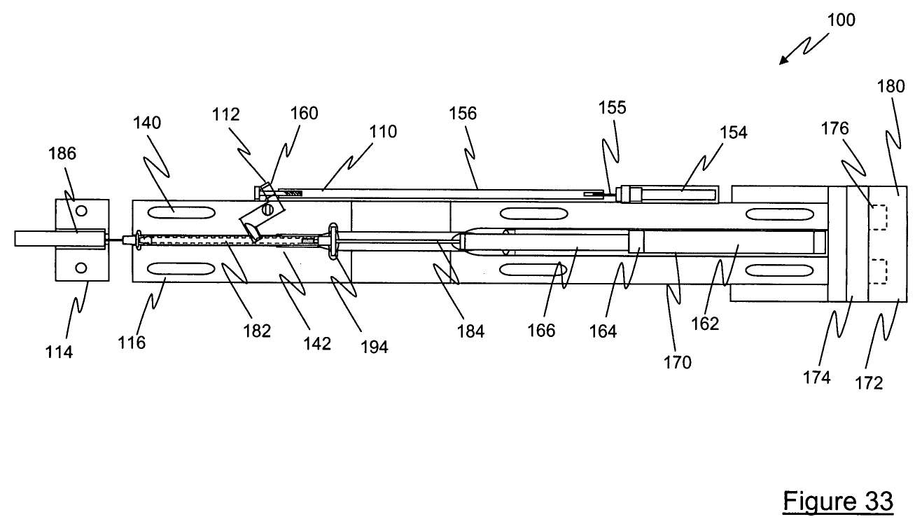

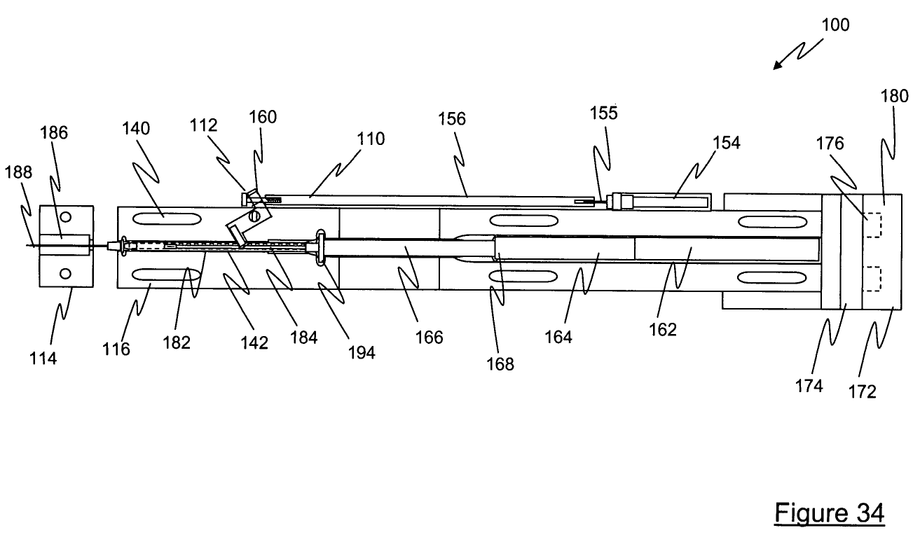

Syringe handling device 100 may be operated, as shown in block 208 and FIG. 33, such that decapper actuation device 108 is configured between the extended configuration 178 and the retracted configuration 180. This causes barrel cradle 116 to move away from needle cradle 114 such that needle cap 186 pushes against channel lip 117 and becomes disassociated with syringe needle 188. Syringe handling device 100 may be disposed, as shown in block 210, such that syringe needle 188 is communicated with a desired container, such as a vial. Syringe handling device 100 may be operated, as shown in block 212 and FIG. 34, such that plunger actuation device 106 is configured between the retracted configuration 170 and the extended configuration 168. As discussed above, as plunger actuation device 106 is configured from the retracted configuration 170 into the extended configuration 168, plunger actuator applies a force to plunger actuation member 164 in the direction of plunger mechanism 166 causing plunger mechanism 166 to move in the direction of syringe 182. As plunger mechanism 166 moves in the direction of syringe 182, plunger mechanism 166 causes syringe plunger 184 to traverse the length of syringe barrel cavity 192 dispensing the substance contained within syringe barrel cavity 192 out of syringe needle 188 and into the container. The vial may then be disposed within a shielded ‘hot cell’ for later use.

It should be appreciated that syringe handling device 100 may include at least one of plunger actuation device 106, decapper actuation device 108 and/or lock actuation device 110 and may be controlled via a processing device communicated with the syringe handling device 100 via any method suitable to the desired end purpose, such as a wireless connection and/or a wired connection. It is further contemplated that syringe nest 104 may be a standalone device without device base 102, such that needle cradle 114 is movably associated with barrel cradle 116. Additionally, syringe 182 may be disposed within syringe nest 104 manually or remotely via a controlled device. Moreover, the method 200 of FIG. 28 may be performed in any order, in part or in whole.

It should further be appreciated that syringe handling device 100 may be constructed of any material or combination of materials, in whole or in part, suitable to the desired end purpose, such as a Delrin or a Ultra High Molecular Weight (UHMW) Polypropylene material. Moreover, construction materials should be chosen such that the counting rate of the mounted syringe will be minimally affected when it is lowered into the dose calibrator.

Although lock actuation device 110, plunger actuation device 106 and decapper actuation device 108 are shown as being a pneumatic device, it should be appreciated that lock actuation device 110, plunger actuation device 106 and/or decapper actuation device 108 may be any type of actuation device suitable to the desired end purpose, such a hydraulic device and/or an electric device and/or a mechanical device and/or any combination thereof.

In accordance with an exemplary embodiment, processing of FIG. 28 may be implemented through a processing device operating in response to a computer program. In order to perform the prescribed functions and desired processing, as well as the computations therefore (e.g., the execution of fourier analysis algorithm(s), the control processes prescribed herein, and the like), the controller may include, but not be limited to, a processor(s), computer(s), memory, storage, register(s), timing, interrupt(s), communication interfaces, and input/output signal interfaces, as well as combinations comprising at least one of the foregoing. For example, the controller may include signal input signal filtering to enable accurate sampling and conversion or acquisitions of such signals from communications interfaces. It is also considered within the scope of the invention that the processing of FIG. 28 may be implemented by a controller located remotely from the processing device.

Moreover, in accordance with an exemplary embodiment, the above embodiment(s) can be embodied in the form of computer-implemented processes and apparatuses for practicing those processes. The above can also be embodied in the form of computer program code containing instructions embodied in tangible media, such as floppy diskettes, CD-ROMs, hard drives, or any other computer-readable storage medium, wherein, when the computer program code is loaded into and executed by a computer, the computer becomes an apparatus for practicing the invention. Existing systems having reprogrammable storage (e.g., flash memory) can be updated to implement the invention. The above can also be embodied in the form of computer program code, for example, whether stored in a storage medium, loaded into and/or executed by a computer, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments configure the microprocessor to create specific logic circuits.

While the invention has been described with reference to an exemplary embodiment, it should be understood by those skilled in the art that various changes, omissions and/or additions may be made and equivalents may be substituted for elements thereof without departing from the spirit and scope of the invention. In addition, many modifications may be made to adapt a particular situation or material to the teachings of the invention without departing from the scope thereof. Therefore, it is intended that the invention not be limited to the particular embodiment disclosed as the best mode contemplated for carrying out this invention, but that the invention will include all embodiments falling within the scope of the appended claims. Moreover, unless specifically stated any use of the terms first, second, etc. do not denote any order or importance, but rather the terms first, second, etc. are used to distinguish one element from another.

1. A syringe handling device for handling a syringe having a syringe needle, a syringe barrel defining a syringe cavity and a syringe plunger at least partially disposed within the syringe cavity, the syringe handling device comprising: a syringe support device, wherein said syringe support device includes at least one of a needle support structure and a barrel support structure; and at least one actuation device, wherein said at least one actuation device includes at least one of a decapper actuation device, a plunger actuation device and a lock actuation device.

2. The syringe handling device of claim 1, wherein said barrel support structure defines a barrel channel having a forward barrel channel and an aft barrel channel, wherein said forward barrel channel includes a forward barrel channel diameter and wherein said aft barrel channel includes an aft barrel channel diameter, said aft barrel channel diameter being larger than said forward barrel channel diameter.

3. The syringe handling device of claim 2, wherein said barrel support structure further defines a plunger channel communicated with said barrel channel.

4. The syringe handling device of claim 2, wherein said lock actuation device includes a locking mechanism movably disposed adjacent said barrel channel, wherein when said lock actuation device is actuated, said locking mechanism is disposed to cover at least a portion of said barrel channel.

5. The syringe handling device of claim 1, wherein said plunger actuation device includes a plunger mechanism movably associated with said plunger actuation device, wherein said plunger actuation device is disposed such that when the syringe is associated with the syringe support device, said plunger mechanism is in communication with the syringe plunger such that actuation of said plunger actuation device causes the syringe plunger to controllably traverse at least a portion of the syringe cavity.

6. The syringe handling device of claim 1, wherein said lock actuation device includes a locking mechanism movably associated with said syringe support device such that when said lock actuation device is actuated, said locking mechanism interacts with at least one of the syringe support device and the syringe to securingly associate the syringe with at least one of the barrel support structure and the needle support structure.

7. The syringe handling device of claim 1, wherein the syringe includes a syringe cap associated with the syringe needle and wherein said decapper actuation device includes a decapper mechanism associated with said syringe support device such that when said decapper actuation device is actuated, said decapper mechanism causes at least one of said barrel support structure and said needle support structure to move relative to the other of said at least one of said barrel support structure and said needle support structure to interact with said syringe cap to cause said syringe cap to be disassociated from the syringe needle.

8. The syringe handling device of claim 1, wherein the syringe includes a syringe cap associated with the syringe needle and wherein said decapper actuation device includes a decapper mechanism associated with said syringe support device such that when said decapper actuation device is actuated, said decapper mechanism interacts with at least one of the syringe and said syringe cap to cause the syringe cap to be disassociated from the syringe needle.

9. The syringe handling device of claim 1, wherein said at least one actuation device is at least one of a pneumatic device and an electromechanical device.

10. The syringe handling device of claim 1, wherein said syringe handling device is at least partially constructed from a non-metallic material.

11. A method for handling a syringe using a syringe handling device, wherein the syringe handling device includes a syringe support structure and at least one of a plunger actuation device and a decapper actuation device, the method comprising:
associating the syringe with the syringe support structure such that the syringe is associated with at least one of the plunger actuation device and the decapper actuation device; and
operating the syringe handling device such that the syringe and at least one of the plunger actuation device and the decapper actuation device interact with each other.

12. The method of claim 11, wherein the syringe includes a syringe needle, a syringe plunger and a syringe barrel defining a syringe cavity, wherein said syringe needle is communicated with said syringe cavity and wherein said syringe plunger is at least partially disposed within said syringe cavity.

13. The method of claim 12, wherein the syringe support structure includes a locking mechanism and at least one of a needle cradle and a barrel cradle and a locking mechanism, said locking mechanism being movably disposed adjacent at least one of said needle cradle and said barrel cradle.

14. The method of claim 13, wherein said associating includes associating the syringe with the syringe support structure such that at least one of said syringe barrel is associated with said barrel cradle and said syringe needle is associated with said needle cradle.

15. The method of claim 11, wherein said operating includes operating the syringe handling device to cause said locking mechanism to securingly interact with said syringe such that said syringe is securely associated with the syringe support structure.

16. The method of claim 11, wherein the syringe includes a needle cap disposed to cover said syringe needle and wherein said operating includes operating the syringe handling device to cause the decapper actuation device to interact with at least one of said needle cap, the syringe and said syringe support structure such that said needle cap is disassociated from said syringe needle.

17. The method of claim 11, wherein operating includes operating the syringe handling device to cause the plunger actuation device to interact with said syringe plunger such that said syringe plunger traverses at least a portion of said syringe cavity to cause any substance contained within said syringe cavity to be dispensed through said syringe needle.

18. The method of claim 11, wherein the syringe support structure includes a locking mechanism and wherein said operating includes operating at least one of said locking mechanism, the plunger actuation device and the decapper actuation device via a processing device.

19. A machine-readable computer program code, the program code including instructions for causing a controller to implement a method for handling a syringe using a syringe handling device, wherein the syringe handling device includes a syringe support structure and at least one of a plunger actuation device and a decapper actuation device, the method comprising:
associating the syringe with the syringe support structure such that the syringe is associated with at least one of the plunger actuation device and the decapper actuation device; and
operating the syringe handling device such that the syringe and at least one of the plunger actuation device and the decapper actuation device interact with each other.

20. The machine-readable computer program code of claim 19, wherein said machine-readable computer program code is encoded onto a storage medium.
